(19) United States
(12) Patent Application Publication
(10) Pub. No.: US 2003/0100066 A1
(43) Pub. Date: May 29, 2003

(54) Yeast transformed with cdna of deer velvet antler for the production of efficient pharmacological ingredients and the preparation of the transformed yeast

(76) Inventor: Hyun-Su Bae, Seoul (KR)

Correspondence Address: Fleshner & Kim, PO Box 221200, Chantilly, VA, 20153-1200, US

(21) Appl. No.: 10/240,797

(22) PCT Filed: May 26, 2001
(86) PCT No.: PCT/KR01/00892

(51) Int. Cl. C12P 21/02; C12N 1/18; C12N 15/74

(52) U.S. Cl. 435/69.1; 435/483; 435/254.2

(57) ABSTRACT

This invention is related to yeast which is transformed to cDNA of deer velvet antler for production of efficacy substance.

Also, This invention contains the step of separate mRNA from deer velvet antler having pharmaceutic effect, the step of processing to cDNA from mRNA, the step of process to cDNA library, and the step of production to yeast strain containing cDNA library.

Therefore, this invention is related to processing method of upper transformed yeast.

### TECHNICAL FIELD

The present invention relates to a yeast transformed with a cDNA of a deer velvet antler for preparing an efficient pharmacological ingredient of a deer velvet antler, and in particular to a method for preparing a transformed yeast based on a step for separating a certain gene having much pharmacological effects, a step for selecting a yeast strain which is proper for preparing an Eukaryotic cell protein and a step for cloning a deer velvet antler gene.

### BACKGROUND ART

A deer velvet antler is a kind of a bone tissue which is reproduced every year in a deer. The above deer velvet antler is generally used as a herb medical material in Asian regions for enhancing a nutrition and immunity. According to the Korean herb medical studies, the above deer velvet antler is known to have excellent medical effects which are experimented and disclosed in many articles. According to the Xinnong medical handbook which is one of the Chinese ancient medical books, the deer velvet antler is known to have a function capable of treating a weak constitution, vigor weakness, womb cold symptom, metrorrhagia and metrostaxis, etc., so that it is good for a nutrition and healthiness, vigor increasing, etc. According to a result of the modern medical studies, it is known that the deer velvet antler is good for a physical growth promotion function, blood formation function, heart enhancing function, a protection function for a damaged liver, a liver tissue reproducing and promotion function, a liver enzyme activation function, a hormone metabolism improving function, an osteoporosis therapy effect, etc.

As the human genome project is nearly completed, a genome information and a gene information of an animal and plant are important together with a coming proteomics era.

### DISCLOSURE OF INVENTION

Accordingly, there is provided a yeast transformed with a cDNA of a deer velvet antler for preparing an efficient pharmacological ingredient and a preparation method of the same which are capable of separating a gene from a deer velvet antler having much pharmacological effects, selecting a yeast strain which is proper for preparing an Eukaryotic protein, expressing a deer velvet antler gene and using a protein for developing a medical supply, food, feed or new medicine based on the expressing deer velvet antler gene.

In order to achieve the above objects, there is provided a preparing method of a yeast transformed with a cDNA of a deer velvet antler for preparing an efficient pharmacological ingredient of a deer velvet antler which includes the steps of separating a mRNA from a tissue cut from a growth point of a deer velvet antler, a step for synthesizing a cDNA from the mRNA, a step for manufacturing library of synthesized cDNA, a step for transforming a yeast with respect to its form and nature based on the preparationd cDNA library, and a step for preparing a yeast transformed with the cDNA based on a pharmacological resistance selection.

As described above, a yeast transformed with cDNA of a deer velvet antler for preparing an efficient pharmacological ingredient and preparation method of same is product much pharmacological ingredient provided pharmacological effect of a deer velvet antler. Therefore, it is used new medicine development which is useful for a protection function for a damaged liver, a liver tissue regeneration and promotion function, a hematopoiesis, a cardiac function, a hormone metabolism improving function, osteoporosis, etc.

### BRIEF DESCRIPTION OF THE DRAWINGS

The present invention will become better understood with reference to the accompanying drawings which are given only by way of illustration and thus are not limitative of the present invention, wherein;

FIG. 1a is a photo illustrating a Cervus elaphus which is known to have an excellent pharmacological effect among the deer according to the present invention;

FIG. 1b is a photo taken at a top end of a grown deer velvet antler according to the present invention;

FIG. 2 is an electrophoresis photo of a plasmid vector (pBluescript) cut using EcoR1 and Not1, where M represents MBI Ferments, and the size of plasmid is 2961 base pairs (bp) according to the present invention; and

FIG. 3 is a graph based on a time passage with respect to a protein concentration and pH which are generated during a yeast cultivation including a cDNA library of a deer velvet antler according to the present invention.

### BEST MODE FOR CARRYING OUT THE INVENTION

For the prefferred embodiment implementations of the present invention, it is preferred to obtain a deer velvet antler in early May through mid-May. At this time, the size of the inserted cDNA is preferably 0.5 through 1.5 kb.

The preferred embodiments of the present invention will be explained in detail.

1. Material and Method

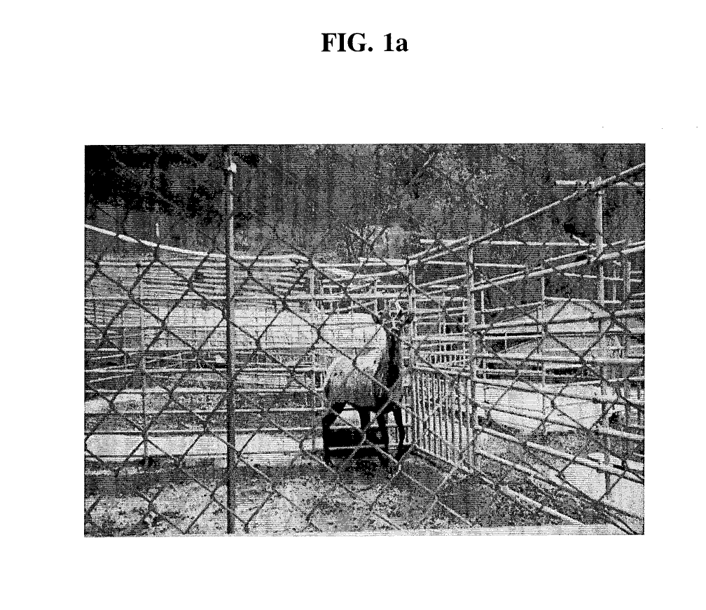

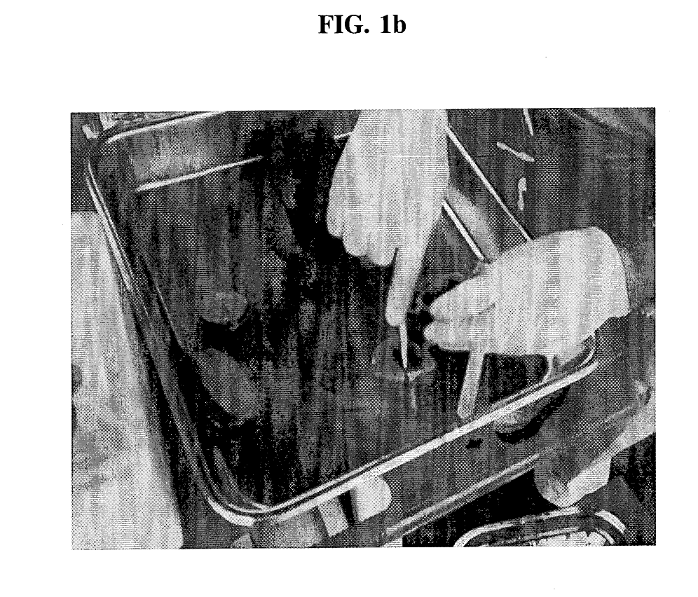

The deer velvet antler was obtained from a Cervus alaphus as shown in FIG. 1. A Gibco BRL product was used for a cell cultivation, RNA separation and cDNA synthesis test sample. In addition, a Qagen was used as a mRNA separation test sample. An Invitrogen product was used as a test sample related to a yeast expression. The test sample above the degree of a molecular living creature was used for other test samples.

(1) Test Sample Selection and Collection

The deer velvet antler was cut in early may through mid-May in which the antler of the cervus elaphus which is known to have an excellent pharmacological effect in the deer, has the most prime growing activity. The cut tissue was quickly frozen using a dry ice and ethanol. Thereafter, the growth tissue was separated, and then an experiment was performed at a storage temperature of −80° C.

(2) Total RNA Separation from the Growth Tissue

The trizole (Gibco RBL) of 3 ml and the separated growth tissue of 3 g was inserted into a conical tube of 50 ml and then was homogenized using a homogenizer. A trizole of 27 ml was added thereto and was well mixed and stored at a room temperature for 5 minutes. The above mixture was divided into four tubes by 7.5 ml, respectively. A chloroform of 1.5 ml was added to each tube and then was stored at a room temperature for three minutes and was centrifugal-processed at 12,000 rpm for 15 minutes. The upper fluid was moved to a new tube of 3 ml, and the isophanol of 3.75 ml was added to each tube and was stored at a temperature of 4° C. for 10 minutes and was centrifugal-processed at 12,000 rpm for 10 minutes. Thereafter, the upper fluid was removed. The total RDA which was a deposit was rinsed using 75% ethanol of 7.5 ml and then was centrifugal-processed at 7,5000 rpm for 5 minutes and then the upper fluid was removed. The total RNA was dried at a room temperature and was molten into a 225 μl DEPC processed distilled water.

(3) mRNA Separation from the Total RNA

mRNA was separated from the thusly separated total RNA using Oligotex mRNA midikit (Qagen product).

(4) cDNA Synthesis and Cloning from the Separated mRNA

The cDNA synthesis from mRNA was performed using a cDNA synthesis kit fabricated by the Gibco BRL company. The experiment was performed based on a slightly modified method.

In a first strand cDNA synthesis, Not I primer-adapter (0.5 μl) 2 μl was inserted into mRNA (2 μg) 9 μl and was well mixed and was reacted at a temperature of 70° C. for 10 minutes for thereby changing the RNA second structure. The mixture was cooled in ice and was inserted into a mixture of 5× first strand buffering liquid 4 μl, 0.1M DTT 2 μl and 10 mM dNTP of 1 μl and was well mixed and was reacted at a temperature of 37° C. for 2 minutes. In addition, a superscriptase II of 2 μl was added to the resultant mixture and then was reacted at a temperature of 37° for 60 minutes and was stored in ice for 2 minutes.

In a second strand cDNA synthesis, a DEPC process distilled water of 91 μl, a second strand buffering solution of 30 μl, a 10 mM dNTP mixture of 3 μl, Escherichia coli (E. coli) DNA ligase (10U/μl), a Escherichia coli (E. coli) DNA polymerase (10U/μl), bacteria RNase H(2U/μl) of 1 μl were added to the above reaction solution and then were reacted at a temperature of 16° C. for 2 hours. Thereafter, a T4 DNA polymerase 2 μl (10U) was added to the resultant mixture and was reacted for 5 minutes. 0.5M EDTA of 10 μl was added to the reacted solution. cDNA was extracted using phenol-chloroform and was molten in the DEPC processed distilled water.

EcoR1-BstX1 adapter (Invitrogen) was ligated to the synthesized cDNA and was cut using Not1. Thereafter, cDNA having Not1 and EcoR1 sequence was prepared at both ends of the cDNA strand. A cDNA having a desired size was separated and obtained through a gel filtering column. A vector (pBluescript, pPICZ, pYES3/CT, etc.) having Not1 and EcoR1 sequence was ligated to a multiple cloning portion. The Escherichia coli (ElectroMAX DH5α, Gibco BRL) was transformed with respect to its form and nature using the electroporation method. The plasmid vector including cDNA was amplified.

(5) Yeast Strain Preparation Including cDNA Library

The yeast strain was transformed with respect to its form and nature based on the electroporation method using the plasmid vector including a deer velvet antler. The yeast cells which were increased in order for O.D₌₆₀₀ to have 1.3 through 1.5, was changed to a competent cell for thereby implementing an electroporation. The above cells of 80 μl were transformed with respect to its form and nature to a vector of 5 μg including a deer velvet antler cDNA (1.5 kV, 25 μF, 200 Ω, 0.2 cm cuvette, Gene-Pulser, BioRad). The colonies were increased in order for O.D₌₆₀₀ to have 4 in a BMG culture (100 mM potassium phosphate, 1.34 YNB, 4×10⁻⁵ biotin, 1% Glycerol) including a 1% casamino acid and was centrifugal-processed to 1,500 through 3,000×g and was diluted and cultured in a BMM culture (100 mM potassium phosphate, 1.34 YNB, 4×10⁻⁵ biotin, 0.5% Glycerol) including 1% casamino acid in order for O.D₌₆₀₀ to have 1 for thereby expressed protein. The expressed protein was checked using SDS-PAGE.

2. Results

(1) Test Sample Selection and Collection

The antler of the Cervus elaphus (FIG. 1) which was known to have an excellent pharmacological effect in the deer, was cut and obtained in Early May through mid-May in which the growth point was most active, and a undifferentiated tissue (fibroblast) near the growth point was obtained.

(2) mRNA Separation and cDNA Synthesis from Growth Tissue

The total RNA was extracted from the separated growth tissue of 3 g, and mRNA was separated from the total RNA, and the cDNA was synthesis-processed. EcoR1-BstX1 adapter was ligated to the synthesis-processed cDNA and was cut using Not1. Thereafter, the cDNA having Not1 and EcoR1 sequence was formed at both ends of cDNA. In order to obtain a cDNA having a desired size, the cDNA was separated through a gel filtering column for thereby obtaining seven fractions. The fractions #7, #8 and #9 having highest concentrations were obtained by comparing the form and nature transformation of each fraction and were ligated to the vector (pBluescript). The E. coli (DH5α) was transformed with respect to its form and nature using the plasmid vector E. coli based on the electroporation for hereby preparing a library including a deer velvet antler cDNA. It was checked that the size of the library was 4×10⁵ through 5×10⁵colonies.

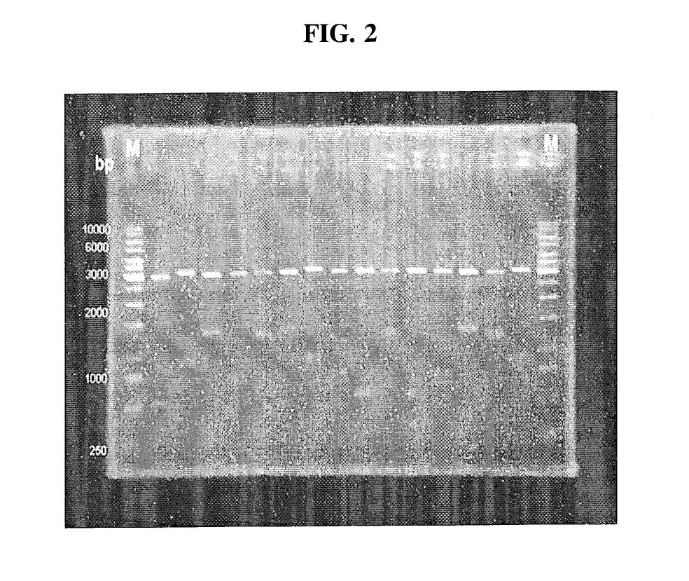

Each colony was obtained for checking the insertion, and the plasmid DNA was separated and cut into EcoR1 and Not1 for comparing the same. As a result of the comparison, it was checked that the insertion of 0.5 through 1.5 kb was inserted (FIG. 2).

(3) Yeast Strain Preparation Including cDNA Library and Efficient Ingredient Analysis Thereof

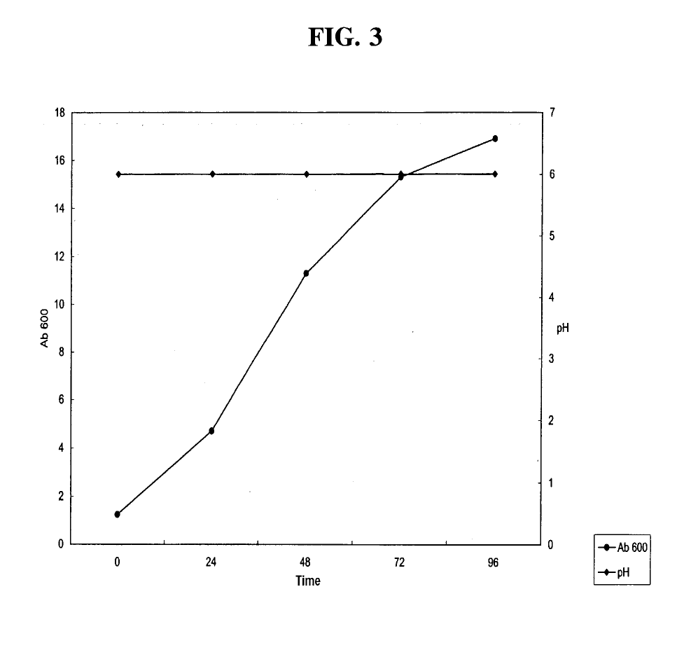

In order to express a cDNA library in a yeast, the plasmid was cut and formed in a linear shape and was inserted into the yeast using an electroporation for thereby implementing a transformation with respect to its form and nature. As a result of selecting the colony which includes a plasmid indicating the resistance with respect to zeocin, 1,000 colonies were preparationd with respect to plasmid DNA of 1 μg. Each colony having a checked insertion was cultured in the BMG culture for increasing the quantity of cells and was moved to the BMM culture and was cultured therein for thereby guiding the preparation of the inserted genes. In addition, the concentration and pH of the protein which was produced during the yeast culture including a cDNA library of a deer velvet antler were checked based on time passage (FIG. 3). Each culture solution was desalted after 4 days, and the expressed protein was checked in 12.5% SDS-PAGE.

In addition, in order to verify the repeating reproducibility of the selected yeast strain, it was deposited to the Korea Federation of Culture Collection (KFCC) on Jan. 20, 2001. The microorganism deposited number report was received from the KFCC on Jan. 31, 2001. The yeast strain was requested to be transformed as a deposited yeast strain based on the Budapest Treaty on May 16, 2001. The deposit receipt number which represents that the yeast strain was deposited to the Korean Culture Center of Microorganism (KCCM) was received (Deposited number: KCCM-10277).

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore total changes and modifications that total within the meets and bounds of the claims, or equivalences of such meets and bounds are therefore intended to be embraced by the appended claims.

Industrial Applicability

As described above, the present invention relates to a yeast transformed with a cDNA of a deer velvet antler for preparing an efficient pharmacological ingredient of a deer velvet antler. In the present invention, it is possible to preparation an efficient ingredient which includes various pharmacological effects of a deer velvet antler because a deer velvet antler ingredient is included in a transformed yeast. Therefore, it is possible to adapt the present invention for a functional food fabrication and feed production using a protein based on the preparation of a deer velvet antler gene. In addition, the present invention may be well adapted to a new medicine development which is useful for a liver protection and liver system reproduction and growth promotion, blood generating function, heart enhancing function, hormone metabolism function improvement, osteoporosis therapy effect, etc. based on an efficient ingredient of a deer velvet antler.

What is claimed is:

1. A preparing method of a yeast transformed with a cDNA of a deer velvet antler for preparing an efficient pharmacological ingredient of a deer velvet antler, comprising:
a step for separating a mRNA from a tissue cut from a growth point of a deer velvet antler;
a step for synthesizing a cDNA from the mRNA;
a step for manufacturing library of synthesized cDNA:
a step for transforming a yeast with respect to its form and nature based on the preparationd cDNA library; and
a step for preparing a yeast transformed with the cDNA based on a pharmacological resistance selection.

2. A method according to claim 1, wherein tissue is obtained from a deer velvet antler in early May through mid-May in which a growth of the deer velvet antler is most active.

3. A method according to claim 1, wherein the size of the inserted cDNA is 0.5 through 1.5 kb.

4. A yeast transformed based on the preparing method of claim 1.
